(19) United States
(12) Patent Application Publication
(10) Pub. No.: US 2005/0064000 A1
(43) Pub. Date: Mar. 24, 2005

(54) Preparation containing 2-methyl-1,3-propanediol

(75) Inventors: Rainer Kropke, Schenefeld (DE); Jens Nielsen, Henstedt-Ulzburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/915,045

(22) Filed: Aug. 9, 2004

Related U.S. Application Data
(63) Continuation of application No. PCT/EP03/01203, filed on Feb. 7, 2003.

(30) Foreign Application Priority Data
Feb. 8, 2002 (DE) 102 05 192.5

(51) Int. Cl. A61K 7/46; A61K 6/00; A61K 7/00

(52) U.S. Cl. 424/401; 512/1

(57) ABSTRACT

The invention includes an aqueous, alcoholic, or aqueous-alcoholic cosmetic or dermatological preparation comprising (a) 2-methyl-1,3-propanediol in a concentration of from 0.5 to 10% by weight and (b) perfume oil in a concentration of from 0.01 to 10.0% by weight, wherein the preparation includes a dispersed phase and an external phase, and wherein the difference in the refractive indices of the dispersed phase and the external phase is greater than 0.003. The invention also includes a method of adding fragrance to a preparation and a method of improving the solubility of perfume in a preparation comprising adding 2-methyl-1,3-propanediol. The invention also includes methods of cleaning, deodorizing, or imparting fragrance by applying the preparation to the skin or to household items.

### CROSS-REFERENCE TO RELATED APPLICATIONS

This is a continuation application of PCT/EP03/01203, filed Feb. 7, 2003, which is incorporated herein by reference in its entirety, and also claims the benefit of German Priority Application No. 102 05 192.5, filed Feb. 8, 2002.

### FIELD OF THE INVENTION

The present invention relates to aqueous and/or alcoholic cosmetic or dermatological preparations comprising 2-methyl-1,3-propanediol and perfume oil with a difference in the refractive indices of dispersed phase and external phase of greater than 0.003, to the process for the preparation and to the use thereof.

### BACKGROUND OF THE INVENTION

Fragrance and perfume materials have been used by man since primeval times to influence his environment. It may be assumed that there already existed a large number of natural sources of fragrance in the environment of early man which were used in connection with satisfying primitive needs. With the discovery of fire, the use of fragrances was given considerable impetus and a new dimension in the life of man. Incense offering is the oldest form of worship, and the burning of certain parts of plants is one of the oldest methods of producing a pleasant odour. The fragrance develops here “per fumum”, through smoke.

The perfuming of the human body has also been known since time immemorial. Egyptians, Persians and Scythians used resins and plant oils enriched with fragrances for embalming their dead. In the old testament, there is a complete recipe for a balm oil under Exodus 30 in the second book of Moses.

Perfumes are produced nowadays from natural plant and animal fragrances and also from chemically synthesized compounds. Plant fragrances are usually obtained by steam distillation in the form of essential oils. One example is eugenol obtained from the oil of cloves. For readily volatile, temperature-sensitive fragrances, for example the oil from jasmine flowers, enfleurage has been used as extraction method since time immemorial for obtaining the “absolu de chassis”. Sensitive citrus oils are obtained by expression from fruit peels. In addition, conventional extraction processes, for example for obtaining resinoids from balsams and resins or benzoins from certain types of lichen and moss, are also used.

Animal fragrances are obtained primarily from glandular excretions of certain types of animal, such as musk, civet or beaver. These glandular secretions, which are used by the animals primarily for marking territory, are only used in dilute form due to their strong odour.

Due to conservation and protection of species, and also because of the high price, many natural fragrances are produced semisynthetically or completely synthetically. The chemical synthesis also allows the development of new, unknown scent notes.

Fragrances and the perfume oils produced from them are used in virtually all cosmetic preparations and in detergents and household cleaners. They serve to mask the intrinsic odour of constituents of these preparations and, in the case of cosmetics and detergents, the intrinsic odour of their user [W. Umbach (Ed.): Kosmetik, Entwicklung, Herstellung und Anwendung kosmetischer Mittel [Cosmetics, development, preparation and use of cosmetic compositions], 2nd ed., Thieme Verlag, Stuttgart, 1995].

Fragrances and the perfume oils produced from them are in most cases non-polar, oil-soluble compounds and mixtures which are only sparingly soluble, or not soluble at all, in water, alcohol or aqueous-alcoholic solutions. In order to be able to incorporate and stabilize perfume oils in such polar preparations, solubility promoters are added to these preparations. These solubility promoters in most cases have an amphiphilic structure and resemble the emulsifiers and surfactants. As a rule, ethoxylated carbohydrates or fatty acid derivatives, for example, polyoxyethylene(20) sorbitan monolaurate (Tween 20) or polyoxyethyleneoxypropylene monostearate (Atlas G-2162), are used for solubilizing perfume oils.

The use of solubility promoters in cosmetic or dermatological preparations is in itself not harmful. Nevertheless, solubility promoters, like ultimately any chemical substance, can cause allergic reactions or reactions based on hypersensitivity of the user in individual cases. Undesired secondary effects which may arise in sensitive people are skin irritations, for example. For this reason, their concentration in cosmetic or dermatological preparations should be kept as low as possible or be avoided altogether.

It is also a disadvantage of the prior art that complex mixtures of solubility promoters often have to be used in order to arrive at stable cosmetic preparations. The suitable compositions of these mixtures can in most cases only be discovered as a result of much experimentation, which makes the development of these preparations laborious and expensive. Moreover, a large number of solubility promoters are not pure substances, but, as a result of the preparation, mixtures, thereby further increasing the problems associated with product compatibility and product stability.

### SUMMARY OF THE INVENTION

It was therefore the object of the present invention to overcome the disadvantages of the prior art and to develop new solubility promoters with which stable, readily compatible, aqueous and/or alcoholic cosmetic perfume preparations can be prepared in a simple manner.

Many aqueous and/or alcoholic cosmetic preparations are transparent. However, consumers often associate products with a low active ingredient content with water-clear, transparent solutions. By contrast, slightly cloudy products are associated with a high active ingredient content and high cosmetic or dermatological performance.

It was therefore a further object of the present invention to impart a cloudy, translucent appearance to the cosmetic preparations according to the invention.

Surprisingly, the objects are achieved by aqueous and/or alcoholic cosmetic or dermatological preparations comprising

a) 2-methyl-1,3-propanediol in a concentration of from 0.5 to 30% by weight,

b) perfume oil in a concentration of from 0.01 to 10.0% by weight, in each case based on the total weight of the preparation, with a difference in the refractive indices of dispersed phase and external phase of greater than 0.003.

These preparations are characterized by high compatibility and performance. Moreover, they can be used to formulate a large number of extremely stable cosmetic or dermatological preparations in a simple manner.

Although WO 00/33804 describes perfume compositions which can also comprise 2-methyl-1,3-propanediol, this specification was unable to point the way to the present invention since it only describes transparent and not cloudy, translucent preparations.

It is particularly advantageous when the preparations according to the invention comprise

a) 2-methyl-1,3-propanediol in a concentration of from 2 to 10% by weight,

b) perfume oil in a concentration of from 0.1 to 3.0% by weight in each case based on the total weight of the preparation.

In addition, it is advantageous according to the invention when the difference in the refractive indices of dispersed phase and external phase is greater than 0.01.

### DETAILED DESCRIPTION OF THE PREFERRED EMBODIMENTS

According to the invention, the weight ratio of 2-methyl-1,3-propanediol to perfume oil in the preparations according to the invention is advantageously 1:1 to 100:1 and particularly preferably 5:1 to 50:1.

The alcohol which is particularly preferred according to the invention is ethanol.

The aqueous and/or alcoholic cosmetic or dermatological preparations according to the invention can of course comprise further cosmetic or dermatological active ingredients, auxiliaries, and additives depending on the intended use. The use of the preparations according to the invention for purposes other than cosmetic or dermatological purposes, for example as detergent, cleaner, household cleaner or disinfectant in the compositions customary for these agents is also in accordance with the invention, the list of these uses in no way intended to be limiting.

According to the invention, besides water, an aqueous solution according to the invention can also comprise other ingredients, for example alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol and glycerol.

The preparations according to the invention can advantageously comprise thickeners. These thickeners may advantageously be chosen from the group of gums, polysaccharides, cellulose derivatives, sheet silicates, polyacrylates and other polymers.

The gums include saps from plants or trees which harden in the air and form resins, or extracts from aquatic plants. From this group, for the purposes of the present invention, gum arabic, carob flour, tragacanth, karaya, guar gum, pectin, gellan gum, carrageen, agar, algins, chondrus, xanthan gum, for example, can be chosen advantageously.

Also advantageous is the use of derivatized gums, such as, for example hydroxypropyl guar (Jaguar® HP 8).

The polysaccharides and polysaccharide derivatives include, for example, hyaluronic acid, chitin and chitosan, chondroitin sulphates, starch and starch derivatives.

The cellulose derivatives include, for example, methylcellulose, carboxymethylcellulose, hydroxyethylcellulose, hydroxypropylmethylcellulose.

The sheet silicates include naturally occuring and synthetic clay earths, such as, for example, montmorillonite, bentonite, hectorite, laponite, magnesium aluminium silicates such as Veegum®. These can be used as such or in modified form, such as, for example, stearylalkonium hectorite.

In addition, silica gels can also be used advantageously.

The polymers according to the invention also include, for example, polyacrylamides (Seppigel 305), polyvinyl alcohols, PVP, PVP/VA copolymers, polyglycols. Polyacrylates which are advantageous according to the invention are polymers of acrylic acid, in particular those which are chosen from the group of so-called carbomers or carbopols (Carbopol® is actually a registered trade mark of the Noveon Company). Polyacrylates are compounds of the general structural formula 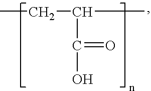
whose molecular weight can be between about 400 000 and more than 4 000 000. The group of polyacrylates also includes acrylate-alkyl acrylate copolymers, for example those which are characterized by the following structure: 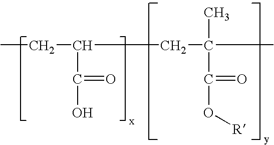
Here, R′ is a long-chain alkyl radical and x and y are numbers which symbolize the respective stoichiometric fraction of the particular comonomers. These polyacrylates are also advantageous for the purposes of the present invention.

Advantageous carbopols are, for example, the grades 907, 910, 934, 940, 941, 951, 954, 980, 981, 1342, 1382, 2984 and 5984 and also the ETD (easy-to-disperse) grades 2001, 2020, 2050, it being possible for these compounds to be present individually or in any combinations with one another.

Particular preference is given to Carbopol® 981, 1382 and ETD 2020 (either individually or in combination).

Also advantageous for the purposes of the present invention are the copolymers of C₁₀-₃₀-alkyl acrylates and one or more monomers of acrylic acid, or methacrylic acid or esters thereof which are comparable to the acrylate-alkyl acrylate copolymers. The INCI name for such compounds is “Acrylates/C10-30 Alkyl Acrylate Crosspolymer”. Those available under the trade name Pemulen TR1 and Pemulen TR2 from B. F. Goodrich Company are particularly advantageous.

Terpolymers for example of vinylpyrrolidone, dimethylaminopropylmethacryl-amide and quaternized alkyldimethylaminopropylmethacrylamide, can also be used advantageously according to the invention.

According to the invention, apart from the above-mentioned substances, the compositions optionally comprise the additives customary in cosmetics, for example perfume, dyes, antimicrobial substances, refatting agents, complexing and sequestering agents, pearlizing agents, further plant extracts, vitamins, active ingredients, preservatives, bactericides, self-tanning agents, depigmentation agents, pigments which have a colouring effect, softening, moisturizing or humectant substances, or other customary constituents of a cosmetic or dermatological formulation, such as polymers, foam stabilizers, electrolytes, organic solvents or silicone derivatives.

An additional content of antioxidants is generally preferred. According to the invention, favourable antioxidants which may be used are all antioxidants which are customary or suitable for cosmetic or dermatological applications.

According to the invention, the preparations according to the invention can advantageously comprise small amounts of preservatives approved for foods. Preservatives approved in food technology which can also be used advantageously for the purposes of the present invention are listed below with their E numbers.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na salt |
| E 216 | N-propyl p-hydroxybenzoate |
| E 217 | N-propyl p-hydroxybenzoate Na salt |
| E 218 | Methyl p-hydroxybenzoate |
| E 219 | Methyl p-hydroxybenzoate Na salt |
| E 220 | Sulphur dioxide |
| E 221 | Sodium sulphite |
| E 222 | Sodium hydrogensulphite |
| E 223 | Sodium disulphite |
| E 224 | Potassium disulphite |
| E 226 | Calcium sulphite |
| E 227 | Calcium hydrogensulphite |
| E 228 | Potassium hydrogensulphite |
| E 230 | Biphenyl (diphenyl) |
| E 231 | Orthophenylphenol |
| E 232 | Sodium orthophenylphenolate |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also advantageous are preservatives or preservative auxiliaries customary in cosmetics, such as dibromodicyanobutane (2-bromo-2-bromomethylglutarodinitrile), phenoxyethanol, 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol.

The preparations according to the invention can advantageously comprise one or more washing-active anionic, cationic, amphoteric, or nonionic surfactants. It is particularly advantageous to choose the washing-active surfactant or surfactants according to the invention from the group of surfactants which have an HLB value of more than 25, those which have an HLB value of more than 35 being particularly advantageous.

Particularly advantageous washing-active anionic surfactants for the purposes of the present invention are acylamino acids and salts thereof, such as
acyl glutamates, in particular sodium acyl glutamate
sarcosinates, for example myristoyl sarcosine, TEA lauroyl sarcosinate, sodium lauroyl sarcosinate and sodium cocoyl sarcosinate,
   sulphonic acids and salts thereof, such as
acyl isethionates, e.g. sodium/ammonium cocoyl isethionate,
sulphosuccinates, for example dioctyl sodium sulphosuccinate, disodium laureth sulphosuccinate, disodium lauryl sulphosuccinate and disodium undecyleneamido MEA sulphosuccinate
   and sulphuric esters, such as
alkyl ether sulphates, for example sodium, ammonium, magnesium, MIPA, TIPA laureth sulphate, sodium myreth sulphate and sodium C₁₂₋₁₃ pareth sulphate,
alkyl sulphates, for example, sodium, ammonium and TEA lauryl sulphate.

Particularly advantageous washing-active cationic surfactants for the purpose of the present invention are quaternary surfactants. Quaternary surfactants comprise at least one N atom which is covalently bonded to 4 alkyl or aryl groups. Benzylalkonium chloride, alkylbetaine, alkylamidopropylbetaine and alkylamidopropylhydroxysultaine are advantageous.

Particularly advantageous washing-active amphoteric surfactants for the purposes of the present invention are
acyl/dialkylethylenediamines, for example sodium acyl amphoacetate, disodium acyl amphodipropionate, disodium alkyl amphodiacetate, sodium acyl amphohydroxypropylsulphonate, disodium acyl amphodiacetate and sodium acyl amphopropionate.

Particularly advantageous washing-active nonionic surfactants for the purposes of the present invention are
alkanolamides, such as cocamide MEA/DEA/MIPA,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers and alkyl polyglycosides such as lauryl glucoside, decyl glycoside and cocoglycoside.
   Further advantageous anionic surfactants are
taurates, for example sodium lauroyl taurate and sodium methylcocoyl taurate,
ether carboxylic acids, for example sodium laureth-13 carboxylate and sodium PEG-6 cocamide carboxylate,
phosphoric esters and salts, such as, for example DEA oleth-10 phosphate and dilaureth-4 phosphate,
alkylsulfonates, for example sodium cocomonoglyceride sulphate, sodium C₁₂₋₁₄ olefinsulphonate, sodium lauryl sulphoacetate and magnesium PEG-3 cocamide sulphate.
   Further advantageous amphoteric surfactants are
N-alkylamino acids, for example aminopropylalkylglutamide, alkylaminopropionic acid, sodium alkylimidodipropionate and lauroamphocarboxyglycinate.
   Further advantageous nonionic surfactants are alcohols.
   Further suitable anionic surfactants for the purposes of the present invention are also
acyl glutamates, such as di-TEA paimitoyl aspartate and sodium caprylic/capric glutamate,
acylpeptides, for example palmitoyl hydrolyzed milk protein, sodium cocoyl hydrolyzed soya protein and sodium/potassium cocoyl hydrolyzed collagen and carboxylic acids and derivatives, such as
for example lauric acid, aluminium stearate, magnesium alkanolate and zinc undecylenate,
ester carboxylic acids, for example calcium stearoyl lactylate, laureth-6 citrate and sodium PEG-4 lauramide carboxylate,
alkylarylsulphonates.
   Further suitable cationic surfactants for the purposes of the present invention are also
alkylamines,
alkylimidazoles and
ethoxylated amines.

Further suitable nonionic surfactants for the purposes of the present invention are also amine oxides, such as cocoamidopropylamine oxide.

Preparations for the purposes of the present invention may preferably comprise at least one UV-A, UV-B and/or broadband filter substance.

Advantageous UV filter substances for the purposes of the present invention are, for example, sulphonated, water-soluble UV filters, such as:
phenylene-1,4-bis(2-benzimidazyl)-3,3′-5,5′-tetrasulphonic acid and its salts, particularly the corresponding sodium, potassium or triethanolammonium salts, in particular the phenylene-1,4-bis(2-benzimidazyl)-3,3′-5,5′-tetrasulphonic acid bis-sodium salt with the INCI name Bisimidazylate (CAS No.: 180898-37-7), which is available, for example, under the trade name Neo Heliopan AP from Haarmann & Reimer;
salts of 2-phenylbenzimidazole-5-sulphonic acid, such as its sodium, potassium or its triethanolammonium salt, and the sulphonic acid itself with the INCI name Phenylbenzimidazole Sulphonic Acid (CAS. No. 27503-81-7), which is available, for example, under the trade name Eusolex 232 from Merck or under Neo Heliopan Hydro from Haarmann & Reimer;
1,4-di(2-oxo-10-sulpho-3-bornylidenemethyl)benzene (also: 3,3′-(1,4-phenylene-dimethylene) bis(7,7-di methyl-2-oxobicyclo[2.2. 1]hept-1 -ylmethanesulphonic acid) and salts thereof (particularly the corresponding 10-sulphato compounds, in particular the corresponding sodium, potassium or triethanolammonium salt), which is also referred to as benzene-1,4-di(2-oxo-3-bornylidenemethyl-10-sulphonic acid). Benzene-1,4-di(2-oxo-3-bornylidenemethyl-10-sulphonic acid) has the INCI name Terephtalidene Dicamphor Sulphonic Acid (CAS No.: 90457-82-2) and is available, for example, under the trade name Mexoryl SX from Chimex;
sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)sulphonic acid and salts thereof.

The list of specified UV filters which can be used for the purposes of the present invention is not of course intended to be limiting.

The preparations according to the invention advantageously comprise the substances which absorb UV radiation in the UV-A and/or UV-B range, in a total amount of, for example, 0.1% by weight to 30% by weight, preferably 0.5 to 20% by weight, in particular 1.0 to 15.0% by weight, in each case based on the total weight of the emulsions, in order to provide cosmetic preparations which protect the hair and/or the skin from the entire range of ultraviolet radiation.

According to the invention, large amounts of acidic aluminium salts and/or aluminium/zirconium salts can be incorporated stably into the preparation. 5 to 40% by weight, in particular 10 to 20% by weight, of aluminium chlorohydrate and/or aluminium/zirconium chlorohydrate can be stably incorporated into the preparations according to the invention. Here, the described concentration ranges refer to the so-called active contents of the antiperspirant complexes: in the case of the aluminium compounds, to anhydrous complexes, in the case of the aluminium/zirconium compounds, to anhydrous and buffer-free complexes. The buffer used is usually glycine.

The list below of antiperspirant active ingredients to be used advantageously is in no way intended to be limiting:

Aluminium salts (of the empirical formula [Al₂(OH)ₘClₙ], where m+n=6):

aluminium salts, such as aluminium chloride AlCl₃, aluminium sulphate Al₂(SO₄)₃
aluminium chlorohydrate [Al₂(OH)₅Cl]×H₂O
standard Al complexes: Locron L (Clariant), Chlorhydrol (Reheis), ACH-303 (Summit), Aloxicoll L (Giulini).
activated Al complexes: Reach 501 (Reheis), AACH-324 (Summit)
aluminium sesquichlorohydrate [Al₂(OH)_{4.5}Cl_{1.5}]×H₂O
standard Al complexes: Aluminum Sesquichlorohydrate (Reheis), ACH-308 (Summit), Aloxicoll 31L (Giulini)
activated Al complexes: Reach 301 (Reheis)
aluminium dichlorohydrate [Al₂(OH)₄Cl₂]×H₂O
   Aluminium-zirconium salts:
aluminium/zirconium trichlorohydrex glycine [Al₄Zr(OH)₁₃Cl₃]×H₂O×Gly
standard Al/Zr complexes: Rezal 33GP (Reheis), AZG-7164 (Summit), Zirkonal P3G (Giulini)
activated Al/Zr complexes: Reach AZZ 902 (Reheis), AAZG-7160 (Summit), Zirkonal AP3G (Giulini)
aluminium/zirconium tetrachlorohydrex glycine [Al₄Zr(OH)₁₂Cl₄]×H₂O×Gly standard Al/Zr complexes: Rezal 36G (Reheis), AZG-368 (Summit), Zirkonal L435G (Giulini)
activated Al/Zr complexes: Reach AZP 855 (Reheis), AAZG-6313-15 (Summit), Zirkonal AP4G (Giulini)
aluminium/zirconium pentachlorohydrex glycine [Al₈Zr(OH)₂₃Cl₅]×H₂O×Gly
standard Al/Zr complexes: Rezal 67 (Reheis), Zirkonal L540 (Giulini)
activated Al/Zr complexes: Reach AZN 885 (Reheis); and
aluminium/zirconium octachlorohydrex glycine [Al₈Zr(OH)₂₀Cl₈]×H₂O×Gly.

However, glycine-free aluminium/zirconium salts may also likewise be advantageous.

In this connection, the use of the antiperspirant active ingredient from the raw material classes of aluminium salts and aluminium/zirconium salts should not be limited to the standard commercial mostly aqueous solutions, such as, for example, Locron L (Clariant), but it may also be advantageous to use likewise standard commercial anhydrous powders of the same raw materials by incorporating them into the claimed formulations, such as, for example, Locron P (Clariant).

Furthermore, however, it may also be advantageous to use special aluminum salts and aluminum/zirconium salts which are supplied as glycol complexes to improve the solubility.

Further advantageous antiperspirant active ingredients are based not on aluminium or zirconium, but on other metals, such as, for example, beryllium, titanium, hafnium.

In this connection, the list of antiperspirant active ingredients which can be used should, however, not be limited to metal-containing raw materials, but compounds which contain non-metals such as boron, and those which are assigned to the field of organic chemistry, such as, for example, anticolinergics, are also advantageous. In this sense, polymers which may either be metal-containing or metal-free are also advantageous.

The process for the preparation of an aqueous and/or alcoholic cosmetic or dermatological preparation according to the invention is also in accordance with the invention. This is characterized in that the perfume oil is firstly dissolved in 2-methyl-1,3-propanediol and then the other constituents are added to the preparation.

Also in accordance with the invention is the use of 2-methyl-1,3-propanediol as solubility promoter in aqueous and/or alcoholic cosmetic or dermatological preparations.

Also in accordance with the invention is the use of the aqueous and/or alcoholic cosmeticor dermatological preparations according to the invention as toner, toilet water, aftershave, deodorant, antiperspirant, face-cleansing lotion, make-up remover, hair tonic, hair-setting and -shaping composition, bathing preparation, foam bath or shower bath, insect repellent, depigmentation agent.

The use of aqueous and/or alcoholic cosmetic or dermatological preparations as lotion, solution, impregnation, tincture or spray is also in accordance with the invention.

The examples below serve to illustrate the present invention without limiting it. Unless stated otherwise, all amounts, fractions and percentages are based on the weight and the total amount or on the total weight of the preparations.

Aqueous and/or alcoholic preparation
(Toner, eye make-up remover, aftershave)

| | 1 | 2 | 3 | 4 | 5 |
|---|---|---|---|---|---|
| PEG 40 hydrogenated castor | 1.0 | — | 3.0 | 1.0 | 0.5 |
| oil | | | | | |
| PEG 8 | 2.0 | — | 2.0 | — | 5.0 |
| Polyethylene glycol(21) | — | 0.5 | — | — | 0.5 |
| stearyl ether | | | | | |
| Glycerol | — | 5.0 | — | 15.0 | 5 |
| 2-Methyl-1,3-propanediol | 5.0 | 2.5 | 10.0 | 8.5 | 1.0 |
| Citric acid | 0.2 | 0.1 | 0.2 | 0.3 | 1.0 |
| Perfume | 0.1 | 5.0 | 3.0 | 2.0 | 1.0 |
| Ethanol | 2.0 | — | 5.0 | — | 10.0 |
| Methylparaben | 0.4 | 0.15 | 0.05 | 0.3 | 0.4 |
| Propylparaben | 0.3 | 0.4 | 0.25 | 0.15 | — |
| Iodopropynyl butylcarbamate | — | — | 0.05 | — | 0.1 |
| Water | ad | ad | ad | ad | ad |
| | 100 | 100 | 100 | 100 | 100 |

1. An aqueous, alcoholic, or aqueous alcoholic cosmetic or dermatological preparation comprising
a) 2-methyl-1,3-propanediol in a concentration of from 0.5 to 30% by weight, based on the total weight of the preparation, and
b) perfume oil in a concentration of from 0.01 to 10.0% by weight, based on the total weight of the preparation,
wherein the preparation includes a dispersed phase and an external phase, and
wherein the difference in the refractive indices of the dispersed phase and the external phase is greater than 0.003.

2. The preparation as claimed in claim 1, further comprising at least one compound selected from the group consisting of cosmetic or dermatological active ingredients, auxiliaries, and additives.

3. The preparation as claimed in claim 1, wherein the weight ratio of 2-methyl-1,3-propanediol to perfume oil is from 1:1 to 100:1.

4. The preparation as claimed in claim 1, wherein the weight ratio of 2-methyl-1,3-propanediol to perfume oil is from 5:1 to 50:1.

5. The preparation as claimed in claim 1, wherein the 2-methyl-1,3-propanediol is present in a concentration of from 2 to 10% by weight and the perfume oil is present in a concentration of from 0.1 to 3.0% by weight.

6. The preparation as claimed in claim 1, wherein the difference in the refractive indices of the dispersed phase and the external phase is greater than 0.01.

7. The preparation as claimed in claim 1, further comprising at least one alcohol.

8. The preparation as claimed in claim 7, wherein the at least one alcohol includes ethanol.

9. The preparation as claimed in claim 1, further comprising at least one compound selected from the group consisting of aluminium salts and aluminium/zirconium salts.

10. The preparation as claimed in claim 1, further comprising at least one UV filter.

11. The preparation as claimed in claim 1, further comprising at least one washing-active surfactant.

12. The preparation as claimed in claim 11, wherein the at least one washing-active surfactant includes at least one surfactants selected from the group consisting of acylamino acids and salts thereof, sulphonic acids and salts thereof; sulphuric esters; cationic quaternary surfactants; acyl/dialkylethylenediamine amphoteric surfactants; alkanolamides; esters which are formed by esterification of carboxy asxz lic acids with ethylene oxide, glycerol, sorbitan or other alcohols; ethoxylated alcohols; ethoxylated lanolin; ethoxylated polysiloxanes; propoxylated POE ethers; and alkyl polyglycosides.

13. The preparation as claimed in claim 1, wherein the preparation is in the form of a lotion, solution, impregnation, tincture, or spray preparation.

14. A method of adding fragrance to an aqueous, alcoholic, or aqueous-alcoholic cosmetic or dermatological preparation, comprising dissolving perfume oil in 2-methyl-1,3-propanediol to form a perfume base and combining said perfume base with additional ingredients to form a preparation.

15. A method for improving the solubility of perfume in an aqueous, alcoholic, or aqueous-alcoholic cosmetic or dermatological preparation comprising adding 2-methyl-1,3-propanediol to the preparation.

16. A method of cleaning, deodorizing, or imparting fragrance on the skin comprising applying to the skin an aqueous, alcoholic, or aqueous alcoholic cosmetic or dermatological preparation, comprising:
a) 2-methyl-1,3-propanediol in a concentration of from 0.5 to 30% by weight, based on the total weight of the preparation, and
b) perfume oil in a concentration of from 0.01 to 10.0% by weight, based on the total weight of the preparation,
wherein the preparation includes a dispersed phase and an external phase, and wherein the difference in the refractive indices of the dispersed phase and the external phase of the preparation prior to application to the skin is greater than 0.003.

17. The method as claimed in claim 16, wherein the weight ratio of 2-methyl-1,3-propanediol to perfume oil in the preparation is from 5:1 to 50:1.

18. The method as claimed in claim 16, wherein the 2-methyl-1,3-propanediol is present in the preparation in a concentration of from 2 to 10% by weight and the perfume oil is present in the preparation in a concentration of from 0.1 to 3.0% by weight.

19. The method as claimed in claim 16, wherein the difference in the refractive indices of the dispersed phase and the external phase in the preparation prior to application to the skin is greater than 0.01.

20. A method of cleaning, disinfecting, or deodorizing a household item comprising applying to the item a preparation comprising
a) 2-methyl-1,3-propanediol in a concentration of from 0.5 to 30% by weight, based on the total weight of the preparation, and
b) perfume oil in a concentration of from 0.01 to 10.0% by weight, based on the total weight of the preparation,
wherein the preparation includes a dispersed phase and an external phase, and
wherein the difference in the refractive indices of the dispersed phase and the external phase of the preparation is greater than 0.003.
